SEQUENCE LISTING (26 sequences)

SEQ ID NO: 1 - protein, 37 aa, Artificial Sequence
  P31
RSCQQQIQQAQQLSSCQQYLKQRVQSEEGEDQISQRE

SEQ ID NO: 2 - protein, 22 aa, Artificial Sequence
  P3
RSCQQQIQQAQQLSSCQQYLKQ

SEQ ID NO: 3 - protein, 15 aa, Artificial Sequence
  P1
RVQSEEGEDQISQRE

SEQ ID NO: 4 - protein, 17 aa, Artificial Sequence
  P2
RAEAFQTAQALPGLCRI

SEQ ID NO: 5 - protein, 10 aa, Artificial Sequence
  P3b
LSSCQQYLKQ

SEQ ID NO: 6 - protein, 12 aa, Artificial Sequence
  P3a
RSCQQQIQQAQQ

SEQ ID NO: 7 - protein, 6 aa, Artificial Sequence
  P3x
RSCQQQ

SEQ ID NO: 8 - protein, 8 aa, Artificial Sequence
  P3y
IQQAQQLS

SEQ ID NO: 9 - protein, 8 aa, Artificial Sequence
  P3z
SCQQYLKQ

SEQ ID NO: 10 - protein, 23 aa, Artificial Sequence
  peptidomimetic
  MISC_FEATURE (12)..(12): X is ornithine, N-lipid tail-glycine, N-neutral  aromatic moiety-glycine or N-acid aromatic moiety- glycine
RSCQQQIQQAQXQLSSCQQYLKQ

SEQ ID NO: 11 - protein, 25 aa, Artificial Sequence
  peptidomimetic
  MISC_FEATURE (1)..(1): X is ornithine, N-lipid tail-glycine, N-neutral aromatic moiety-glycine or N-acid aromatic moiety- glycine
  MISC_FEATURE (13)..(13): X is ornithine, N-lipid tail-glycine, N-neutral aromatic moiety-glycine or N-acid aromatic moiety- glycine
  MISC_FEATURE (25)..(25): X is ornithine, N-lipid tail-glycine, N-neutral aromatic moiety-glycine or N-acid aromatic moiety- glycine
XRSCQQQIQQAQXQLSSCQQYLKQX

SEQ ID NO: 12 - protein, 24 aa, Artificial Sequence
  peptidomimetic
  MISC_FEATURE (1)..(1): X is ornithine, N-lipid tail-glycine, N-neutral aromatic moiety-glycine or N-acid aromatic moiety- glycine
  MISC_FEATURE (22)..(22): X is ornithine, N-lipid tail-glycine, N-neutral aromatic moiety-glycine or N-acid aromatic moiety- glycine
  misc_feature (24)..(24): Xaa can be any naturally occurring amino acid
XRSCQQQIQQAQQLSSCQQYLKQX

SEQ ID NO: 13 - protein, 9 aa, Artificial Sequence
  peptidomimetic
  MISC_FEATURE (5)..(5): X is ornithine, N-lipid tail-glycine, N-neutral aromatic moiety-glycine or N-acid aromatic moiety- glycine
IQQAXQQLS

SEQ ID NO: 14 - protein, 11 aa, Artificial Sequence
  peptidomimetic
  MISC_FEATURE (1)..(1): X is ornithine, N-lipid tail-glycine, N-neutral aromatic moiety-glycine or N-acid aromatic moiety- glycine
  MISC_FEATURE (6)..(6): X is ornithine, N-lipid tail-glycine, N-neutral aromatic moiety-glycine or N-acid aromatic moiety- glycine
  MISC_FEATURE (11)..(11): X is ornithine, N-lipid tail-glycine, N-neutral aromatic moiety-glycine or N-acid aromatic moiety- glycine
XIQQAXQQLSX

SEQ ID NO: 15 - protein, 10 aa, Artificial Sequence
  peptidomimetic
  MISC_FEATURE (1)..(1): X is ornithine, N-lipid tail-glycine, N-neutral aromatic moiety-glycine or N-acid aromatic moiety- glycine
  MISC_FEATURE (10)..(10): X is ornithine, N-lipid tail-glycine, N-neutral aromatic moiety-glycine or N-acid aromatic moiety- glycine
XIQQAQQLSX

SEQ ID NO: 16 - protein, 22 aa, Artificial Sequence
  peptidomimetic
  MISC_FEATURE (1)..(1): X is N-(3-guanidinopropyl)glycine
  MISC_FEATURE (7)..(7): X is N-(sec-butyl)glycine
  MISC_FEATURE (10)..(10): X is N-methylglycine
  MISC_FEATURE (13)..(13): X is N-isobutylglycine
  MISC_FEATURE (20)..(20): X is N-isobutylglycine
  MISC_FEATURE (21)..(21): X is N-(4-aminobutyl)glycine
XSCQQQXQQXQQXSSCQQYXXQ

SEQ ID NO: 17 - protein, 22 aa, Artificial Sequence
  peptidomimetic
  MISC_FEATURE (1)..(1): X is N-(3-guanidinopropyl)glycine
  MISC_FEATURE (7)..(7): X is N-(sec-butyl)glycine
  MISC_FEATURE (13)..(13): X is N-isobutylglycine
  MISC_FEATURE (21)..(21): X is N-(4-aminobutyl)glycine
XSCQQQXQQAQQXSSCQQYLXQ

SEQ ID NO: 18 - protein, 22 aa, Artificial Sequence
  peptidomimetic
  MISC_FEATURE (1)..(1): X is N-(3-guanidinopropyl)glycine
  MISC_FEATURE (10)..(10): X is N-methylglycine
  MISC_FEATURE (21)..(21): X is N-(4-aminobutyl)glycine
XSCQQQIQQXQQLSSCQQYLXQ

SEQ ID NO: 19 - protein, 22 aa, Artificial Sequence
  peptidomimetic
  MISC_FEATURE (1)..(1): X is N-(3-guanidinopropyl)glycine
  MISC_FEATURE (21)..(21): X is N-(4-aminobutyl)glycine
XSCQQQIQQAQQLSSCQQYLXQ

SEQ ID NO: 20 - protein, 22 aa, Artificial Sequence
  peptidomimetic
  MISC_FEATURE (1)..(1): X is N-(3-guanidinopropyl)glycine
  MISC_FEATURE (20)..(20): X is N-isobutylglycine
  MISC_FEATURE (21)..(21): X is N-(4-aminobutyl)glycine
XSCQQQIQQAQQLSSCQQYXXQ

SEQ ID NO: 21 - protein, 22 aa, Artificial Sequence
  peptidomimetic
  MISC_FEATURE (1)..(1): X is N-(3-guanidinopropyl)glycine
  MISC_FEATURE (10)..(10): X is N-methylglycine
XSCQQQIQQXQQLSSCQQYLKQ

SEQ ID NO: 22 - protein, 22 aa, Artificial Sequence
  peptidomimetic
  MISC_FEATURE (10)..(10): X is N-methylglycine
  MISC_FEATURE (21)..(21): X is N-(4-aminobutyl)glycine
RSCQQQIQQXQQLSSCQQYLXQ

SEQ ID NO: 23 - protein, 22 aa, Artificial Sequence
  peptidomimetic
  MISC_FEATURE (10)..(10): X is N-methylglycine
RSCQQQIQQXQQLSSCQQYLKQ

SEQ ID NO: 24 - protein, 8 aa, Artificial Sequence
  peptidomimetic
  MISC_FEATURE (1)..(1): X is N-(sec-butyl)glycine
  MISC_FEATURE (4)..(4): X is N-methylglycine
  MISC_FEATURE (7)..(7): X is N-isobutylglycine
XQQXQQXS

SEQ ID NO: 25 - protein, 8 aa, Artificial Sequence
  peptidomimetic
  MISC_FEATURE (1)..(1): X is N-(sec-butyl)glycine
  MISC_FEATURE (7)..(7): X is N-isobutylglycine
XQQAQQXS

SEQ ID NO: 26 - protein, 8 aa, Artificial Sequence
  peptidomimetic
  MISC_FEATURE (4)..(4): X is N-methylglycine
IQQXQQLS

(19) United States
(12) Patent Application Publication
(10) Pub. No.: US 2022/0153786 A1
(43) Pub. Date: May 19, 2022

(54) PEPTIDES FOR PREVENTING BIOFILM FORMATION

(71) Applicants: UNIVERSITE DE RENNES 1, RENNES CEDEX (FR); Centre national de la recherche scientifique, PARIS (FR); UNIVERSIDADE FEDERAL DO RIO GRANDE DO SUL UFRGS, PORTO ALEGRE (BR)

(72) Inventors: Rafael GOMES VON BOROWSKI, NOVO HAMBURGO - RS (BR); Aline RIGON ZIMMER, PORTO ALEGRE,RIO GRANDE DO SUL (BR); Simone Cristina BAGGIO GNOATTO, PORTO ALEGRE (BR); Alexandre José MACEDO, PORTO ALEGRE (BR); Muriel PRIMON DE BARROS, PORTO ALEGRE (BR); Karine RIGON ZIMMER, PORTO ALEGRE (BR); Reynald GILLET, LE VERGER (FR); Grace GOSMANN, PORTO ALEGRE (US)

(73) Assignees: UNIVERSITE DE RENNES 1, RENNES CEDEX (FR); Centre national de la recherche scientifique, PARIS (FR); UNIVERSIDADE FEDERAL DO RIO GRANDE DO SUL UFRGS, PORTO ALEGRE (BR)

(21) Appl. No.: 17/432,026

(22) PCT Filed: Feb. 20, 2020
(86) PCT No.: PCT/EP2020/054435

§ 371 Date: Aug. 18, 2021

(30) Foreign Application Priority Data
Feb. 20, 2019 (EP) 19305205.7

(51) Int. Cl. C07K 14/415; A61L 27/54; A61L 29/16; C07K 7/08

(52) U.S. Cl. CPC C07K 14/415; A61L 27/54; A61L 2300/404; C07K 7/08; A61L 2300/25; A61L 29/16

(57) ABSTRACT

The present invention relates to the use of a peptide to prevent biofilm formation by microorganisms on a surface, wherein said peptide is
(i) a peptide consisting of 6 to 37 consecutive amino acids from a peptide of sequence SEQ ID NO: 1,
(ii) a peptide of sequence SEQ ID NO: 4, or
(iii) a peptidomimetic of (i) or (ii).

### CROSS REFERENCE TO RELATED APPLICATIONS

The present application is a U.S. National Phase Application under 35 U.S.C. § 371 of International Patent Application No. PCT/EP2020/054435 filed Feb. 20, 2020, which claims priority of European Patent Application No. 19305205.7 filed Feb. 20, 2019. The entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Feb. 20, 2019, is named SequenceListing_2021-08-18_245735-000008.txt and is 11,413 bytes in size.

### FIELD OF THE INVENTION

The present invention relates to peptides for preventing biofilm formation by microorganisms.

### BACKGROUND

Antimicrobial failure is a worldwide challenge, endorsed in a currently global action plan. The lack of novel antibiotics and their inappropriate uses are resulting in an increase of multi-drug tolerant and resistant strains. This process is favored by biofilm development hence microorganisms enclosed in the matrix display up to 1000 times higher antibiotic resistance than the planktonic ones making the biofilm matrix itself a new important target.

Moreover, the biofilm is a complex matrix, composed of extracellular polymeric substance (EPS) wrapping microorganisms communities, irreversibly adhered to a biotic (e.g. tissues and organs) or abiotic (e.g. catheters, prostheses, kitchen or marine utensils) surface. Micro colonies are the beginning of mature biofilm and its structure and composition can react or be adsorbed with external agents mediating the adhesion to surfaces and also promoting physical protection against the antibiotics or the immune system response. Accordingly, adhesion and colonization are required for the establishment of bacterial infection and pathogenesis. Thus, the bacteria may undergo specific molecular changes to establish biofilms and adhering on implanted devices as probes, prostheses, catheters or on damaged tissues, impacting importantly on the patients' denouement and costs of the health system.

In this context, Staphylococcus epidermidis is the most frequently negative coagulase Staphylococcus infection causing disease, being capable to survive on surfaces for months. Staphylococcus epidermidis is an emerging pathogen bacteria and the ability to form biofilms on devices is its major virulence factor. Biofilm has been considered an important virulence factor, which is present in 80% of human infections as endocarditis, osteomyelitis, chronic sinusitis, urethritis, periodontitis, characterizing it as a severe public health problem. The extracellular matrix is a complex physicochemical barrier that represents one of the highest difficulties in prevention or treatment of biofilm. Biofilms are often the cause of difficulty in eradicating bacteria, representing a challenge in different areas, especially medical, odontological, navy and food industry surroundings.

Therefore, the development of antivirulence strategies such as efficient antibiofilm agents is crucial against the current antibiotic crisis.

In particular, there is a need for identifying antiadhesive agents as an alternative strategy to prevent bacterial attaching and infection.

Currently, there is a great interest in the search for new natural bioactive compounds against bacterial biofilms, ubiquitous in natural, clinical and industrial environments, and responsible for the high tolerance and resistance of bacteria to antimicrobial agents. Some non-biocidal strategies that target the related virulence factors have been proposed. In this scenario, peptides and peptidomimetics are rising as important arsenal. However, there are currently no anti-biofilm drugs available.

Thus there is still a need for anti-biofilm strategies targeting biofilm matrix in order to fight multi-drug tolerant and resistant bacteria.

Plant-derived compounds have gained extensive interest in the search for alternatives to control microorganisms. These compounds are widely accepted due to their use in folk medicine for the prevention and treatment of diseases and infections.

The extracts from fruits of Capsicum baccatum var. pendulum, a pepper widely consumed in Brazil, have been shown to present antioxidant, anti-inflammatory, and antifungal properties. However, there is still scarce biological and chemical information about this species.

### SUMMARY

The present invention arises from the unexpected finding by the inventors that peptides isolated from an extract of C. baccatum seeds have potent anti-biofilm activity. More specifically, the inventors have demonstrated herein that peptides derived from 2S sulfur-rich seed storage protein (s.s.p) 2-like from C. baccatum significantly inhibits biofilm formation by microorganisms.

In particular, the inventors have discovered new antibiofilm peptides derived from the red pepper Capsicum baccatum that prevent adhesion, biofilm establishment and maintenance of microorganisms. Importantly, these peptides are non-antibiotic and non-cytotoxic, providing a new alternative to prevent biofilm infections. In particular, the peptides according to the invention prevent bacterial adhesion and biofilm formation of the methicillin resistant S. epidermis and do not show antibiotic activity. These properties evidence their potential for combating antibiotic-tolerant and resistant bacteria, and controlling clinical and industrial problems related to biofilms.

The present invention thus relates to the use of a peptide to prevent biofilm formation by microorganisms on a surface, wherein said peptide is:
(i) a peptide consisting of a fragment of 6 to 37 consecutive amino acid residues, in particular of 8 to 37 consecutive amino acid residues, more particularly of 10 to 37 consecutive amino acids residues from a peptide of sequence SEQ ID NO: 1 (RSCQQQIQQAQQLSSCQQYLKQRVQSEEGEDQISQRE),
(ii) a peptide of sequence SEQ ID NO: 4 (RAEAFQTAQALPGLCRI), or
(iii) a peptidomimetic of (i) or (ii).

It also relates to an isolated peptide consisting of an amino acid sequence selected from the group consisting of:

SEQ ID NO: 2
(RSCQQQIQQAQQLSSCQQYLKQ),
SEQ ID NO: 1
(RSCQQQIQQAQQLSSCQQYLKQRVQSEEGEDQISQRE),
SEQ ID NO: 3
(RVQSEEGEDQISQRE),
SEQ ID NO: 4
(RAEAFQTAQALPGLCRI),
SEQ ID NO: 5
(LSSCQQYLKQ),
SEQ ID NO: 6
(RSCQQQIQQAQQ),
SEQ ID NO: 7
(RSCQQQ),
SEQ ID NO: 8
(IQQAQQLS)
and
SEQ ID NO: 9
(SCQQYLKQ),

or a peptidomimetic thereof.

The present invention also relates to a composition comprising the isolated peptide of the present invention.

The present invention also relates to an ex vivo method for preventing biofilm formation by microorganisms on a device comprising at least one surface, said method comprising the step of coating said at least one surface with the composition of the present invention.

The present invention further relates to a device comprising at least one surface coated with the peptide of the present invention.

The present invention also relates to the peptide of the present invention for use in a method for preventing infections such as endocarditis, osteomyelitis, chronic sinusitis, urethritis or periodontitis in a patient by the prevention of biofilm formation by microorganisms.

### DETAILED DESCRIPTION

### Peptide

The terms “peptide” refers to amino acid sequences of a variety of lengths. In preferred embodiments, the amino acid sequence is a fragment of the peptide of the present invention.

In a particular embodiment, the peptide according to the invention is a peptide consisting of an amino acid sequence selected from the group consisting of:

SEQ ID NO: 2
(RSCQQQIQQAQQLSSCQQYLKQ),
SEQ ID NO: 1
(RSCQQQIQQAQQLSSCQQYLKQRVQSEEGEDQISQRE),
SEQ ID NO: 4
(RAEAFQTAQALPGLCRI)
SEQ ID NO: 3
(RVQSEEGEDQISQRE),
SEQ ID NO: 5
(LSSCQQYLKQ),
SEQ ID NO: 6
(RSCQQQIQQAQQ),
SEQ ID NO: 7
(RSCQQQ),
SEQ ID NO: 8
(IQQAQQLS)
and
SEQ ID NO: 9
(SCQQYLKQ),

or is a peptidomimetic thereof.

In another particular embodiment, the peptide according to the invention is a peptide consisting of an amino acid sequence selected from the group consisting of:

SEQ ID NO: 2
(RSCQQQIQQAQQLSSCQQYLKQ),
SEQ ID NO: 1
(RSCQQQIQQAQQLSSCQQYLKQRVQSEEGEDQISQRE),
SEQ ID NO: 4
(RAEAFQTAQALPGLCRI)
SEQ ID NO: 3
(RVQSEEGEDQISQRE),
SEQ ID NO: 6
(RSCQQQIQQAQQ),
SEQ ID NO: 7
(RSCQQQ),
SEQ ID NO: 8
(IQQAQQLS)
and
SEQ ID NO: 9
(SCQQYLKQ),

or is a peptidomimetic thereof.

In still another particular embodiment, the peptide according to the invention is a peptide consisting of an amino acid sequence selected from the group consisting of:

SEQ ID NO: 2
(RSCQQQIQQAQQLSSCQQYLKQ),
SEQ ID NO: 1
(RSCQQQIQQAQQLSSCQQYLKQRVQSEEGEDQISQRE),
SEQ ID NO: 4
(RAEAFQTAQALPGLCRI)
SEQ ID NO: 3
(RVQSEEGEDQISQRE),
SEQ ID NO: 5
(LSSCQQYLKQ),
and
SEQ ID NO: 6
(RSCQQQIQQAQQ),

or is a peptidomimetic thereof.

In still another particular embodiment, the peptide according to the invention is a peptide consisting of an amino acid sequence selected from the group consisting of:

SEQ ID NO: 2
(RSCQQQIQQAQQLSSCQQYLKQ),
SEQ ID NO: 1
(RSCQQQIQQAQQLSSCQQYLKQRVQSEEGEDQISQRE),
SEQ ID NO: 4
(RAEAFQTAQALPGLCRI)
SEQ ID NO: 3
(RVQSEEGEDQISQRE),
and
SEQ ID NO: 6
(RSCQQQIQQAQQ),

or is a peptidomimetic thereof.

In a preferred embodiment, the peptide according to the invention is a peptide consisting of SEQ ID NO: 2.

In another preferred embodiment, the peptide according to the invention is a peptide of sequence SEQ ID NO: 8.

The term “fragment”, when used herein, refers to a peptide comprising or consisting of an amino acid sequence of at least 6 consecutive amino acid residues, in particular at least 8, more particularly at least 10 consecutive amino acids, and of less than 38 consecutive amino acid residues of the peptide of sequence SEQ ID NO: 1, typically of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 consecutive amino acids residues of the peptide of sequence SEQ ID NO: 1. In a particular embodiment, the peptide of the present invention is a fragment of 6, 8, 10, 11, 12, 13, 14, 15, 16, 17, 18, 19, 20, 21 or 22 consecutive amino acid residues from a peptide of sequence SEQ ID NO: 1, preferably 8, 10, 11, 12, 13, 14, 15, 16, 17, 18, 19, 20, 21 or 22 consecutive amino acid residues from a peptide of sequence SEQ ID NO: 1, still preferably 10, 12, 15 or 22 consecutive amino acid residues of the protein or polypeptide.

In a particular embodiment, said peptide comprises or consists of an amino acid sequence of at least 6 consecutive amino acids, in particular at least 8 consecutive amino acids, and of less than 22 amino acids residues of the peptide of SEQ ID NO: 2, typically of 6, 7, 8, 9, 10, 11, 12, 13, 14, 15, 16, 17, 18, 19, 20, 21 or 22 consecutive amino acids residues of the peptide of sequence SEQ ID NO: 2. In a particular embodiment, the peptide of the present invention is a fragment of 6, 8, 10, 11, 12, 13, 14, 15, 16, 17, 18, 19, 20, 21 or 22 consecutive amino acid residues from a peptide of sequence SEQ ID NO: 2, preferably 6, 8, 10 or 12 consecutive amino acid residues from a peptide of sequence SEQ ID NO: 2.

The invention also relates to peptidomimetics of the peptides according to the invention. Peptidomimetics refer to synthetic chemical compounds, which have substantially the same structural and/or functional characteristics of the peptides according to the invention. The mimetic can be entirely composed of synthetic, non-natural amino acid analogs, or can be a chimeric molecule including one or more natural amino acids and one or more non-natural amino acid analogs. The mimetic can also incorporate any number of natural amino acid conservative substitutions that do not destroy the mimetic's activity. The phrase “substantially the same”, when used in reference to a mimetic or peptidomimetic, means that the mimetic or the peptidomimetic has one or more activities or functions of the referenced molecule, in particular prevention of biofilm formation. The techniques for developing peptidomimetics are conventional. For example, peptide bonds can be replaced by non-peptide bonds or non-natural amino acids that allow the peptidomimetic to adopt a similar structure, and therefore biological activity, to the original peptide. Further modifications can also be performed by replacing chemical groups of the amino acids with other chemical groups of similar structure. Once a potential peptidomimetic compound is identified, it may be synthesized and its ability to prevent biofilm formation can be assayed. Peptidomimetics can contain any combination of non-natural structural components, which are typically from three structural groups: residue linkage groups other than the natural amine bond (“peptide bond”) linkages; non-natural residues in place of naturally occurring amino acid residues; residues which induce secondary structural mimicry (e.g., beta turn, gamma turn, beta sheet, alpha helix conformation); or other changes which confer resistance to proteolysis.

One or more residues can also be replaced by an amino acid (or peptidomimetic residue) of the opposite chirality. Thus, any amino acid naturally occurring in the L-configuration (which can also be referred to as R or S, depending upon the structure of the chemical entity) can be replaced with the same amino acid or a mimetic, but of the opposite chirality, referred to as the D-amino acid, but which can additionally be referred to as the R- or S-form.

In a particular embodiment, the peptidomimetic according to the invention is an AApeptide. AApeptides refers herein to oligomers of N-acylated-N-aminoethyl-substituted amino acids that are derived from chiral peptide nucleic acid backbones. The chiral side chain is connected to either the α-C or γ-C of the carbonyl group, while acylation is used to introduce the other side chain to the central N. AApeptides have the same backbone lengths and functional group counts, and the same number of nitrogen atoms involved in secondary or tertiary amide bonds than their original peptide counterparts. In addition, they mimic the original amino acid side-chain positions, so they have the same activity.

In another embodiment, the peptidomimetic according to the invention is a peptoid. Peptoids or beta-peptoids are oligomers of N-substituted glycine units. Their side chains extend from the main chain nitrogen rather than from the α-carbon, thus yielding secondary structures including helices, loops and turns. They retain the functionalities and backbone polarity of peptides.

In a particular embodiment, the peptidomimetic according to the invention is a peptidomimetic of a fragment of the peptide of the present invention.

The peptides and peptidomimetics can be produced and isolated from natural products using any method known in the art. Peptides can also be synthesized, whole or in part, using usual chemical methods.

In a particular embodiment, said peptidomimetic is a compound selected from the group consisting of
the compounds of sequence RSCQQQIQQAQXQLSSCQQYLKQ (SEQ ID NO: 10), X₁RSCQQQIQQAQX₂QLSSCQQYLKQX₃ (SEQ ID NO: 11), X₄RSCQQQIQQAQQLSSCQQYLKQX₆ (SEQ ID NO: 12), IQQAX₆QQLS (SEQ ID NO: 13), X₇IQQAX₈QQLSX₆ (SEQ ID NO: 14) and X₁₀IQQAQQLSX₁₁ (SEQ ID NO: 15), wherein
   X, X₁, X₂, X₃, X₄, X₅, X₆, X₇, X₈, X₉, X₁₀ and X₁₁ are independently selected from ornithine, N—Z₁-glycine, N—Z₂-glycine, and N—Z₃-glycine, wherein Z₁ is a lipid tail typically selected from the group consisting of the moieties O—R₁ and PO₃⁻—R₁, wherein R₁ is a saturated or unsaturated O₃—C₂₀ alkyl, in particular a saturated or unsaturated C₆-C₉ alkyl; Z₁ being in particular selected from the group consisting of the moieties of the following formulae:

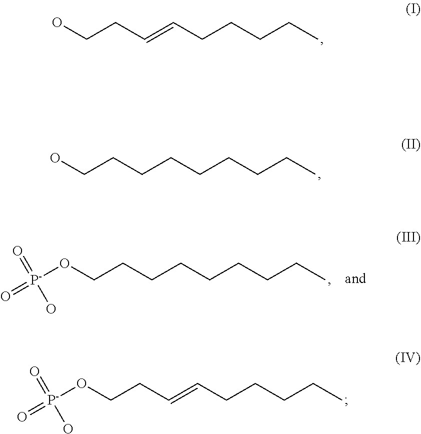

wherein Z₂ is a neutral aromatic moiety typically selected from the group consisting of the moieties of the following formulae:

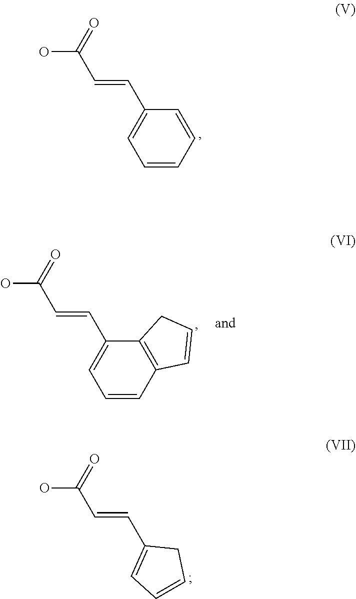

and
wherein Z₃ is an acid aromatic moiety typically selected from the group consisting of the moieties of the following formulae;

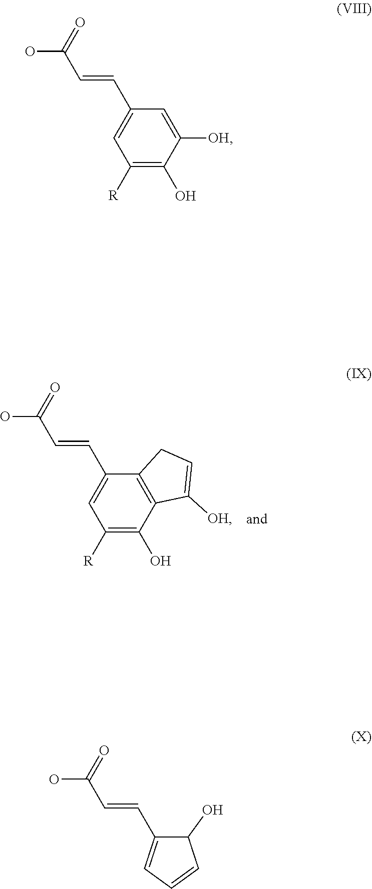

wherein R is H or OH;
the compounds of sequence NArg-SCQQQ-NIle-QQ-NAla-QQ-NLeu-SSCQQY-NLeu-NLys-Q (SEQ ID NO: 16), NArg-SCQQQ-NIle-QQAQQ-NLeu-SSCQQYL-NLys-Q (SEQ ID NO: 17), NArg-SCQQQIQQ-NAla-QQLSSCQQYL-NLys-Q (SEQ ID NO: 18), NArg-SCQQQIQQAQQLSSCQQYL-NLys-Q (SEQ ID NO: 19), NArg-SCQQQIQQAQQLSSCQQY-NLeu-NLys-Q (SEQ ID NO: 20), NArg-SCQQQIQQ-NAla-QQLSSCQQYLKQ (SEQ ID NO: 21), RSCQQQIQQ-NAla-QQLSSCQQYL-NLys-Q (SEQ ID NO: 22), RSCQQQIQQ-NAla-QQLSSCQQYLKQ (SEQ ID NO: 23), NIle-QQ-NAIa-QQ-NLeu-S(SEQ ID NO: 24), NIIe-QQAQQ-NLeu-S(SEQ ID NO: 25), and IQQ-NAla-QQLS (SEQ ID NO: 26), wherein NArg is N-(3-guanidinopropyl)glycine, Nile is N-(sec-butyl)glycine, NAla is N-methylglycine, NLeu is N-isobutylglycine and NLys is N-(4-aminobutyl)glycine;
the α-AA-peptides α-AA-RSCQQQIQQAQQLSSCQQYLKQ of formula (XI)

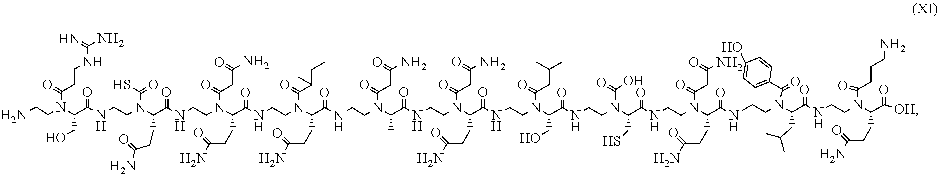 [SMILES: CCC(C)C(=O)N(CCNC(=O)[C@H](CCC(N)=O)N(CCNC(=O)[C@H](CCC(N)=O)N(CCNC(=O)[C@H](CO)N(CCN)C(=O)CCNC(=N)N)C(=O)S)C(=O)CC(N)=O)[C@@H](CCC(N)=O)C(=O)NCCN(C(=O)CC(N)=O)[C@@H](C)C(=O)NCCN(C(=O)CC(N)=O)[C@@H](CCC(N)=O)C(=O)NCCN(C(=O)CC(C)C)[C@@H](CO)C(=O)NCCN(C(=O)O)[C@@H](CS)C(=O)NCCN(C(=O)CC(N)=O)[C@@H](CCC(N)=O)C(=O)NCCN(C(=O)c1ccc(O)cc1)[C@@H](CC(C)C)C(=O)NCCN(C(=O)CCCN)[C@@H](CCC(N)=O)C(=O)O]

α-AA-QQIQQAQQLSSCQQ of formula (XII)

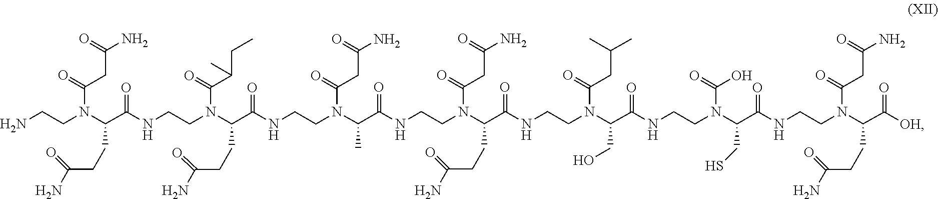 [SMILES: CCC(C)C(=O)N(CCNC(=O)[C@H](CCC(N)=O)N(CCN)C(=O)CC(N)=O)[C@@H](CCC(N)=O)C(=O)NCCN(C(=O)CC(N)=O)[C@@H](C)C(=O)NCCN(C(=O)CC(N)=O)[C@@H](CCC(N)=O)C(=O)NCCN(C(=O)CC(C)C)[C@@H](CO)C(=O)NCCN(C(=O)O)[C@@H](CS)C(=O)NCCN(C(=O)CC(N)=O)[C@@H](CCC(N)=O)C(=O)O]

α-AA-QIQQAQQLSSCQ of formula (XIII)

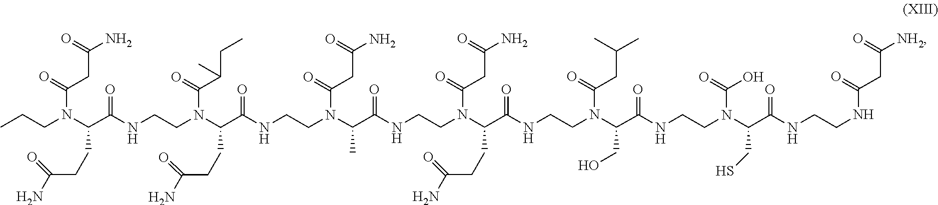 [SMILES: CCCN(C(=O)CC(N)=O)[C@@H](CCC(N)=O)C(=O)NCCN(C(=O)C(C)CC)[C@@H](CCC(N)=O)C(=O)NCCN(C(=O)CC(N)=O)[C@@H](C)C(=O)NCCN(C(=O)CC(N)=O)[C@@H](CCC(N)=O)C(=O)NCCN(C(=O)CC(C)C)[C@@H](CO)C(=O)NCCN(C(=O)O)[C@@H](CS)C(=O)NCCNC(=O)CC(N)=O]

α-AA-IQQAQQLSSC of formula (XIV)

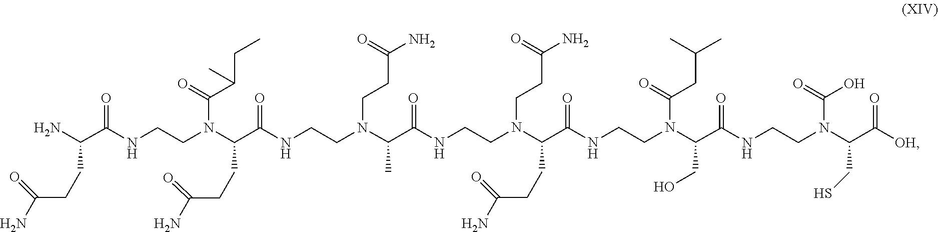 [SMILES: CCC(C)C(=O)N(CCNC(=O)[C@@H](N)CCC(N)=O)[C@@H](CCC(N)=O)C(=O)NCCN(CCC(N)=O)[C@@H](C)C(=O)NCCN(CCC(N)=O)[C@@H](CCC(N)=O)C(=O)NCCN(C(=O)CC(C)C)[C@@H](CO)C(=O)NCCN(C(=O)O)[C@@H](CS)C(=O)O]

and α-AA-IQQAQQLS of formula (XV)

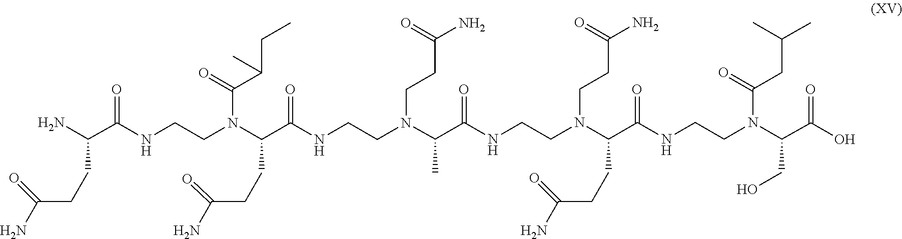 [SMILES: CCC(C)C(=O)N(CCNC(=O)[C@@H](N)CCC(N)=O)[C@@H](CCC(N)=O)C(=O)NCCN(CCC(N)=O)[C@@H](C)C(=O)NCCN(CCC(N)=O)[C@@H](CCC(N)=O)C(=O)NCCN(C(=O)CC(C)C)[C@@H](CO)C(=O)O]

and
the γ-AA-peptides γ-AA-RSCQQQIQQAQQLSSCQQYLKQ of formula (XVI)

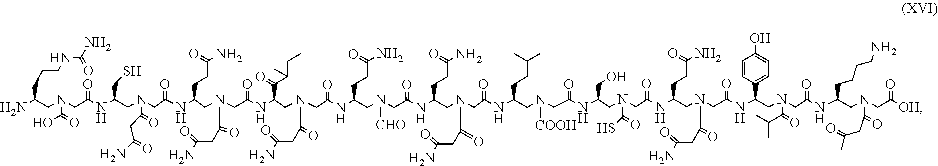 [SMILES: CCC(C)C(=O)[C@@H](CN(CC(=O)N[C@@H](CCC(N)=O)CN(C=O)CC(=O)N[C@@H](CCC(N)=O)CN(CC(=O)N[C@@H](CCC(C)C)CN(CC(=O)N[C@@H](CO)CN(CC(=O)N[C@@H](CCC(N)=O)CN(CC(=O)N[C@H](CN(CC(=O)N[C@@H](CCCCN)CN(CC(=O)O)C(=O)CC(C)=O)C(=O)C(C)C)c1ccc(O)cc1)C(=O)CC(N)=O)C(=O)S)C(=O)O)C(=O)CC(N)=O)C(=O)CC(N)=O)NC(=O)CN(C[C@H](CCC(N)=O)NC(=O)CN(C[C@H](CS)NC(=O)CN(C[C@@H](N)CCCNC(N)=O)C(=O)O)C(=O)CC(N)=O)C(=O)CC(N)=O]

γ-AA-QQIQQAQQLSSCQ of formula (XVII)

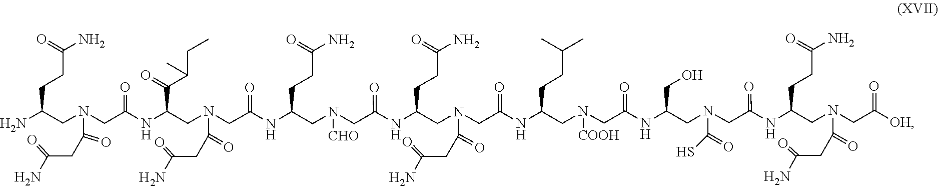 [SMILES: CCC(C)C(=O)[C@@H](CN(CC(=O)N[C@@H](CCC(N)=O)CN(C=O)CC(=O)N[C@@H](CCC(N)=O)CN(CC(=O)N[C@@H](CCC(C)C)CN(CC(=O)N[C@@H](CO)CN(CC(=O)N[C@@H](CCC(N)=O)CN(CC(=O)O)C(=O)CC(N)=O)C(=O)S)C(=O)O)C(=O)CC(N)=O)C(=O)CC(N)=O)NC(=O)CN(C[C@@H](N)CCC(N)=O)C(=O)CC(N)=O]

γ-AA-QIQQAQQLSSC of formula (XVIII)

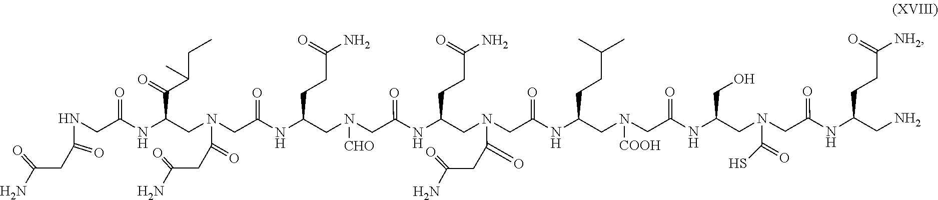 [SMILES: CCC(C)C(=O)[C@@H](CN(CC(=O)N[C@@H](CCC(N)=O)CN(C=O)CC(=O)N[C@@H](CCC(N)=O)CN(CC(=O)N[C@@H](CCC(C)C)CN(CC(=O)N[C@@H](CO)CN(CC(=O)N[C@H](CN)CCC(N)=O)C(=O)S)C(=O)O)C(=O)CC(N)=O)C(=O)CC(N)=O)NC(=O)CNC(=O)CC(N)=O]

γ-AA-IQQAQQLSS of formula (XIX)

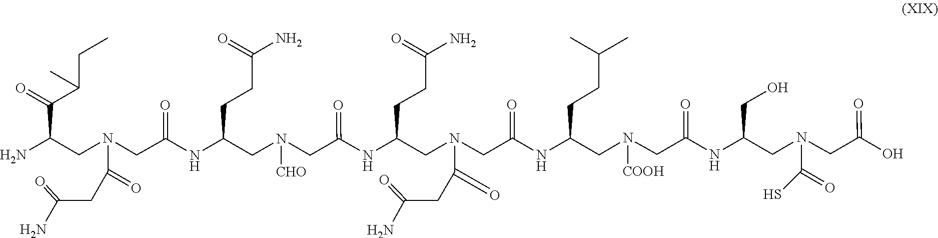 [SMILES: CCC(C)C(=O)[C@H](N)CN(CC(=O)N[C@@H](CCC(N)=O)CN(C=O)CC(=O)N[C@@H](CCC(N)=O)CN(CC(=O)N[C@@H](CCC(C)C)CN(CC(=O)N[C@@H](CO)CN(CC(=O)O)C(=O)S)C(=O)O)C(=O)CC(N)=O)C(=O)CC(N)=O]

and γ-AA-IQQAQQLS of formula (XX)

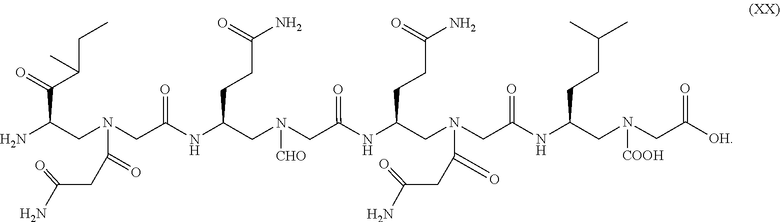 [SMILES: CCC(C)C(=O)[C@H](N)CN(CC(=O)N[C@@H](CCC(N)=O)CN(C=O)CC(=O)N[C@@H](CCC(N)=O)CN(CC(=O)N[C@@H](CCC(C)C)CN(CC(=O)O)C(=O)O)C(=O)CC(N)=O)C(=O)CC(N)=O]

### Biofilm

The inventors have shown that the peptide according to the invention prevent biofilm formation by microorganisms on a surface.

Microbial biofilms are ubiquitous in natural, clinical and industrial environments. Biofilms are complex communities of microorganisms, usually attached to a biotic and/or abiotic surface and encapsulated by a polymeric extracellular matrix of microbial origin. This complicated structure is involved in a multitude of different infections and contribute significantly to the therapeutic failures.

In the sense of the invention, the prevention of biofilm formation by microorganisms refers to the prevention of the establishment and maintenance of biofilm architecture. According to one alternative embodiment, the peptide according to the invention acts on the initial phase of matrix assembly, preventing its functional assembly rather than de-structuring once established. In another embodiment, the peptide interacts with the extracellular matrix and modifies the self-assembly chain, resulting in a less dense nonfunctional matrix that consequently prevents biofilm formation.

In the sense of the present invention, the microorganisms can be any microscopic organisms capable of forming a biofilm, for example bacteria or fungus. In a particular embodiment, the microorganisms may be Staphylococcus epidermidis (S. epidermis), Pseudomonas aeruginosa (P. aeruginosa) or Cryptococcus neoformans (C. neoformans).

In a particular embodiment, the peptide according to the invention prevents biofilm formation without decreasing microorganisms' growth. In the sense of the invention the prevention of the biofilm formation is independent of cell death or growth inhibition. Microorganisms forming the biofilm are typically enclosed in the biofilm matrix and attached to a surface. They express higher physiological and biochemical changes and higher mutation rates than their planktonic forms. In the sense of the invention, the planktonic form of the microorganism refers to its non-adherent form and/or free flowing in suspension.

In a particular embodiment, the peptide according to the present invention does not inhibit planktonic growth of the biofilm-forming organisms. By the inhibition of the planktonic growth is meant the decrease or the end of the planktonic microorganism's growth rate.

Non-antibiotic targets suggest less susceptibility to the development of resistance phenomena than conventional antibiotics because microorganisms suffer a milder evolutionary pressure to generate resistance without the biotic activity. In another embodiment, the peptide according to the present invention prevents biofilm formation and limits the development of microorganisms that are drug resistant and/or tolerant.

In the sense of the invention, drug resistant microorganisms are microorganisms that survive to antimicrobial drugs, e.g. antibiotics, exposure by the acquisition of molecular resistance mechanisms. Drug tolerant microorganisms are microorganisms that survive antimicrobial drugs, e.g. antibiotics, exposure in the absence of acquired molecular resistance mechanisms.

In a particular embodiment, the microorganisms are resistant and/or tolerant to antimicrobial drugs.

In another embodiment, the microorganisms are antibiotic tolerant and/or resistant.

### Surface

In the sense of the invention, the surface on which the biofilm is formed can be any type of surface. The surface may be abiotic or biotic.

Biofilms are often the cause of difficulty in eradicating bacteria, representing a challenge in different areas, especially medical, odontological, navy and food industry surroundings.

The abiotic surface according to the invention may be the surface of a device or of a tooth, or an industrial processing surface. In a particular embodiment, the surface is an immersed surface, preferably a ship's hull, steel, metal, glass, polymers, minerals or ceramic material.

The biotic surface may be host tissue, mucus or a wound.

The present invention also relates a device comprising at least one surface as defined above coated with the peptide according to the invention. The device can be any industrial or medical device as defined above.

According to one advantageous embodiment of the invention, the surface according to the invention is the surface of a medical device.

The medical device according to the invention may be an implanted device or a surgical implant such as probes, prostheses, catheters. In particular, the medical device may be, but is not limited to, a ventricular derivation, an oro-tracheal tubing, a prosthetic cardiac valve, a pacemaker, a urinary catheter or an orthopedic prosthesis. In one particular embodiment, the medical device according to the invention is prone to the formation of biofilm and may lead to a microbial infection. Bacteria are a major concern for people with catheters, probes or other surgical implants because it is known to form biofilms on these devices.

According to another embodiment, the surface according to the invention is the surface of an industrial device.

The industrial device according to the invention may be a food or naval industry related device such as tools, equipment or instruments that have contact with water such as a pipe or a valve.

The peptide of the invention may be under any formulation suitable for coating said device. In a particular embodiment, said at least one surface coated with the peptide of the invention is coated with a hydrogel, such as a diacrylate (PEGDA)-based cross-linked poly(ethyleneglycol) hydrogel, on or into which said peptide of the invention is immobilized, for example by covalent binding on PEGDA).

### Composition

The present invention also relates to a composition comprising the isolated peptide according to the present invention. In particular, the isolated peptide may be the peptide consisting of SEQ ID NO: 2 or the peptide consisting of SEQ ID NO: 8.

Accordingly, the present invention provides a composition comprising an effective amount of the peptide according to the invention. In a particular embodiment, the concentration of the peptide in the composition is comprised between 1 μM and 1 mM, in particular between 1 μM and 100 μM, preferably between 1 μM and 10 μM. In a preferred embodiment, the concentration of the peptide in the composition is 10 μM. In another embodiment, the composition according to the invention is effective from 1 μM.

According to one advantageous embodiment, the composition may be a pharmaceutical composition. The pharmaceutical composition may comprise a pharmaceutically acceptable excipient.

The term “pharmaceutically acceptable” refers to properties and/or substances which are acceptable for administration to a subject from a pharmacological or toxicological point of view.

A pharmaceutical composition according to the invention may be administered in any amount and using any route of administration effective for achieving the desired prophylactic and/or therapeutic effect. The optimal pharmaceutical formulation can be varied depending upon the route of administration and desired dosage. Such formulations may influence the physical state, stability, rate of in vivo release, and rate of in vivo clearance of the administered active ingredient(s).

According to one advantageous embodiment of the invention, the pharmaceutical composition is administrated orally or intravenously. The pharmaceutical composition used within the scope of the invention may be presented in any dosage forms normally used for the oral or intravenous mode of administration. In a particular embodiment, the pharmaceutical composition used within the scope of the invention may be administrated in combination with antibiotics for the prevention and/or treatment of bacterial biofilm-associated infections.

In another particular embodiment, the composition is a coating composition. Said coating composition can comprise any additional compound and/or excipient suitable to improve the use of the composition as coating.

In particular, said coating composition may be a hydrogel, such as a diacrylate (PEGDA)-based cross-linked poly(ethyleneglycol) hydrogel, on or into which said peptide of the invention is immobilized, for example by covalent binding on PEGDA).

Thus the present invention also relates to the peptide according to the invention for use for the prevention of biofilm formation by microorganism in a patient.

The present invention also relates to a method for preventing biofilm formation by microorganisms or infections in a patient in need thereof, said method comprising administering a prophylactically efficient amount of a peptide of the invention or of a composition of the invention to said patient.

The present invention also relates to the use of a peptide of the invention for the manufacture of a medicament intended to prevent infections in a patient.

In particular, the peptide according to the infection may be used for the prevention of infections linked to biofilm in a patient. In a particular embodiment, the infection may be endocarditis, osteomyelitis, chronic sinusitis, a urinary tract infection such as urethritis or an oral infection such as periodontitis.

By “prevention” is meant herein to prevent or slow down the emergence of an infection in a patient.

By “prophylactically efficient amount” is meant herein an amount effective, at dosages and for periods of time necessary, to achieve the desired prophylactic result.

The peptide of the invention may be used under any suitable formulation.

The composition according to the invention may further be used to coat a surface as defined above and thus prevent the formation of a biofilm on said surface.

In a particular embodiment, the composition is an antifouling composition.

By antifouling composition is meant a composition which prevents biofouling, i.e. the accumulation of microorganisms on wetted surface.

The present invention also relates to an ex vivo method for preventing biofilm formation by microorganisms on a surface as defined above, said method comprising the step of coating or modifying said surface with a composition according to the present invention. The coating or modification step may be carried out by any physical, chemical or depositional technique well-known from the skilled person, such as physical or thermal vapor deposition, solution-based processes, polymer-interlayers, biomimicry, induction hardening, nitriding, tufftriding and shot-peening,

The surface may be any type of surface according to the invention. In one embodiment, the method is an ex vivo method for preventing biofilm formation by a microorganisms on a device according to the present invention. In a particular embodiment, the method is an ex vivo method for preventing biofilm formation by a microorganisms on a medical or industrial device according to the present invention.

The present invention will be further illustrated by the figures and examples below.

### BRIEF DESCRIPTION OF THE DRAWINGS

FIGS. 1-4: Antibiofilm activity of 3 peptides.

FIG. 1: Antibiofilm activity of peptides P1, P2, and P3 (capsicumicine) at 1, 10, and 100 μM. Quantification (black bars) of Staphylococcus epidermidis (ATCC 35984) biofilms was done at an optical density of 570 nm after 24 h of peptide exposure, and is shown compared to the bacteria without peptide exposure (Control) and the antibiotic control, 96 μg/mL rifampicin (Rif.). Student's t-test: *, p≤0.05; **, p≤0.01.

FIG. 2: Colony-forming units (CFUs) after 24 h exposure to 10 μM capsicumicine. Instead of the peptide, the control was exposed to vehicle (water), and the result is shown as CFU/mL.

FIG. 3: Biofilm eradication test. Shown are Staphylococcus epidermidis (ATCC 35984) biofilm quantifications at OD₅₇₀ for the bacterial biofilm without peptide exposure (“Growth Control”), after exposure to the rifampicin antibiotic control, and after 24 h treatment with 100 μM capsicumicine.

FIG. 4: Capsicumicine cytotoxicity evaluation in representative human cell lines shown via automated image-based cellular content analysis. Cell counts are presented as residual cell percentages (%) compared to the average of the DMSO control (white), with water control also shown (bar on the right of DMSO control). The three bars on the left show cytotoxic controls (roscovitine, doxycycline, and taxol), while the bars on the right are cells exposed to 10 μM capsicumicine.

FIGS. 5-6: Scanning electron microscopy (SEM) and qRT-PCR analysis.

FIG. 5: SEM images of polystyrene coupons after 1, 4, or 24 h of culture with Staphylococcus epidermidis (ATCC 35984). Top: peptide-less biofilm control; bottom: cultures exposed to 10 μM capsicumicine. Magnification ×500, with insets at ×5,000; scale bars, 10 μm.

FIG. 6: Gene expression (mean log fold changes±standard errors of the means) of the encoding genes involved in S. epidermidis biofilm formation as compared to the planktonic (black) and biofilm controls (grey), with the ssrA gene used as a reference. The group exposed to the peptide (capsicumicine+) is white.

FIGS. 7A-7E: Different microscopic images of Staphylococcus epidermidis (ATCC 35984) biofilm. These images explore the organizational state of the biofilm matrix after 24 h in the presence (right) or absence (control, left) of capsicumicine. (7A) Macroscopic examination using pictures from the bottom of 24-well plates. The “sterile control” shows no bacteria or biofilm formation; the “biofilm control” has homogenous adhered layers of bacteria; and “capsicumicine” has non-adhered bacteria which agglutinate in the peptide's presence. (7B) Transmitted light microscopy images show the biofilm control has many overlapping attached cells surrounded by a matrix next to bacterial clusters, while the capsicumicine-exposed culture has non-adhered but suspended cells that look to be agglutinated. (7C) Confocal fluorescence microscopy images of the fluorescence-free control and the capsicumicine-FITC, featuring a green fluorescent matrix. (7D) Like the previous images, scanning electron microscopy images show the control with dense globular-like matrix features, while the capsicumicine-exposed culture has fibrillary branch-like oligomer structures. (7E) Transmission electron microscopy images shows denser assembled structures in the biofilm matrix control, while the capsicumicine-exposed matrix is less dense and displays thin fibrillary oligomer structures. Arrows indicate the matrices.

FIG. 8: Real-time molecular self-assembly (RTMSA) curves of synthetic staphylococcal matrices. Graph of optical densities (OD₆₀₀) as a function of the time in minutes for the synthetic matrix in the presence of capsicumicine (triangles), the synthetic matrix positive control (circles), and the synthetic matrix PA1 peptide negative control (squares).

### DETAILED DESCRIPTION

### Examples

### Example 1

### Materials and Methods

Peptides. All peptides were synthesized by Biomatik™ and ProteoGenix™ both containing a purity grade greater than 95% in salt suitable for cell culture. Mass spectral and HPLC analysis were provided as quality control. They were all solubilized in ultra-pure sterile water for the assays. Capsicumicine corresponds to the peptide of sequence SEQ ID NO: 2.

Bacterial Strain and growth conditions. Staphylococcus epidermidis ATCC 35984 was grown overnight on blood agar (Thermo Scientific, Oxoid PB5039A) at 37° C. A bacterial suspension of 3×10⁸ colony-forming units (CFU)/mL in tryptone soya broth (TSB, Oxoid Ltd., England, UK) or 0.9% NaCl was used in the assays. Lysogenic broth (LB, Oxoid Ltd., England, UK) agar was used to colony forming units (CFU) assay.

Biofilm formation. All assays were at least performed as technical and biological triplicates using 1, 10 or 100 μM of peptides. Biofilm inhibition: a protocol adapted from Zimmer et al. (2013) Environmental Microbiology 15:2008-2018 and Trentin et al. (2015) Scientific Reports 5:8287, employing crystal violet in 96-well poly(vinyl chloride) microtiter plates (Falcon; Becton Dickinson Labware, Oxnard, Calif.) was used. Briefly, 100 μL of the bacterial suspension, 100 μL of the peptide solution (at different concentrations) or vehicle (to controls) and 50 μL of tryptone soya broth (TSB, Oxoid Ltd., England). Following 37° C. for 1, 4 or 24 h of incubation, the content of the wells was removed and the wells were washed three times with sterile saline. The remaining contents were heat-fixed at 60° C. for 1 h. The adherent biofilm layer formed was stained with 0.4% crystal violet for 15 min at room temperature and then washed three times with distilled water. The stain bound to the cells was solubilized with absolute ethanol (Sigma-Aldrich Co., USA) and absorbance was measured at 570 nm (Powerwave™ XS Plate Reader, BIO-TEK Instruments®, Inc.). The biofilm formation controls represent 100% of biofilm formation. Biofilm eradication: biofilm was pre-formed as described before, during 24 h at 37° C., without treatment. After biofilm formation, the wells were washed to remove the planktonic cells and the peptides solutions and controls were added and incubated for 24 h. The eradication was verified by evaluating the remaining content by crystal violet.

Bacterial growth assays. Microtiter plates: bacterial growth was evaluated by difference between the optical density absorbance at 600 nm measured at the end and the beginning of the incubation time (37° C., 1, 4 or 24 h) in 96-well poly(vinyl chloride) microtiter plates. Rifampicin 16 μg/mL (Sigma-Aldrich Co., USA) was used as a control for bacterial growth inhibition. Colony-forming units (CFU/mL): after incubation (37° C., 24 h) the CFU was calculated to determine bactericidal effect of peptide solution. Untreated growth control was considered 100% of planktonic cells. All assays were at least performed as technical and biological triplicates.

Scanning electron microscopy (SEM): sterile polystyrene coupons (10×4 mm) were co-culture in presence or absence of capsicumicine for 1, 4 and 24 h. After, the coupons were washed with sterile NaCl 0.9% and fixed with glutaraldehyde 2.5%, paraformaldehyde 2%, cacodylate 0.1 M buffer (pH 7.2). Afterwards, they were washed with cacodylate 0.1 M buffer with sucrose 0.2 M and dehydrated with increasing concentrations of ethanol and dehydrated samples were then subjected to Critical Point Drying (Leica EM CPD 300). Finally, they were sputtered with palladium (Leica EM ACE 200) and analyzed by JEOL JSM 7100 F EDS EBSD Oxford microscope, at 10 kV.

Transmission electron microscopy (TEM): all the content of the wells was suitably detached (1, 4 and 24 h cultures in presence or absence of P3), recovered, centrifuged at 10,000 g, 15 min, 4° C. and washed with sterile NaCl 0.9%. Fixation was performed at 4° C. with sodium cacodylate 0.1 M, paraformaldehyde 2%, glutaraldehyde 2.5% and lysine 75 mM. After that, samples were washed with sodium cacodylate 0.1M, sucrose 0.2M and contrasted with osmium tetroxide 1%, potassium ferrocyanide 1.5%. Dehydration was done with gradual solution of ethanol and infiltration with increasing concentration of LR White® resin (Delta Microscopies). Then, LR White® resin inclusion and polymerization were made during 24 h at 60° C. in O₂ absence. Thin sections (80 nm) were collected onto 200 mesh carbon grids, and visualized with a Tecnai Sphera operating at 200 kV (FEI, Eindhoven, Netherlands) equipped with a 4×4 k CCD UltraScan camera (Gatan, Pleasanton, USA).

Confocal fluorescence microscopy (CFM): P3-fluorescein isothiocyanate (capsicumicine-FITC, 10 μM) was used to detect capsicumicine peptide. After incubation (1, 4 and 24 h) all the content of the wells was suitable detached, recovered, centrifuged at 11,000 g, 2 min, 4° C. and washed with sterile NaCl 0.9%. This suspension was visualized directly or after Calcofluor 2 mg/mL (Fluorescent Brightener 28, Sigma) addition. To illustrate bacterial cells permeable by a peptide (control) we used an antimicrobial peptide also labeled with FITC. Those antimicrobial peptides are seen on/in bacteria although not in the extracellular matrix. Images were acquired with Leica SP8 DMI 6000 CS (resonant scanner) confocal microscope with hybrid detector. ImageJ software was used for image analysis.

Quantitative Reverse Transcriptase PCR (qRT-PCR): the RNAs were isolated from planktonic cells (control), biofilm cells (control) or total cells (exposed to capsicumicine at 10 μM), after 4, 24 h cultures. It was applied TRIzol™ Max™ Bacterial RNA Isolation Kit (Invitrogen™) and TURBOT′″ DNase treatment (Ambion®) according to manufacturer's instructions. Concentration and purity of total RNA was spectrophotometrically assessed using SimpliNano™ (Biochrom, USA) and PCR reaction was performed to certify the complete absence of DNA. It was considered 50% of yield for Reverse Transcriptase Reaction (M-MLV, Promega®): 1000 ng of RNA=500 ng cDNA. Then, the inventors used 10 ng of cDNA and 0.2 μM of primers per qRT-PCR reaction, previously verified. Reactional volumes were calculated according to the manufacturer's instructions (SYBR® Select Master Mix, Applied Biosystems Inc; USA). Primers were designed through the Primer3 program (Thermo Fisher® Primers) and according to literature. They were produced by Eurofins Genomic. It was used Applied Biosystems StepOnePlus™ equipment and software. The relative transcript levels were determined by 2^{-ΔΔct} (Livak and Schmittgen (2001) Methods 25:402-408). To validate the selected biofilm encoding genes, the inventors compared planktonic control to biofilm control. They found purposeful differences between biofilm and planktonic controls, as expected.

Real time molecular self-assembly (RTMSA) assay: after checking the starting point (pH) of the assembly reaction for staphylococcal synthetic matrix (Stewart et al. (2015) Sci. Rep. 5:13081), the inventors recorded the optical density (OD=600 nm) in function of the time, each 30 seconds until 30 minutes. Molecular self-assembly reactions were calculated to a final volume of 4 mL, considering 0.3% chitosan (medium molecular weight, 75-85% of deacetylation, Sigma), 0.15% bovine serum albumin (BSA, Sigma), 0.015% lambda DNA (Sigma) in tryptone soya broth (TSB, Oxoid Ltd., England, UK). The concentration of tested peptides was calculated in μM to the final volume of 4 mL (100 μM). Before getting the pH starting point of assembly reaction, a calibration record was done using the same reactional tube containing all reagents (auto zero). The pH adjustments were made using acetic acid and NaOH and the reaction temperature was around 30° C.

Cytotoxicity assay: the assays were performed in a robotic platform (ImPACcell, BIOSIT, Université de Rennes 1) dedicated to multiparameter high-throughput image analysis (HCS: High Content Screening and HCA: High Content Analysis), using 7 different mammalian lines: HuH7, CaCo-2, MDA, HCT116, PC3, NCI-H727 and MCF7. The concentration tested was 10 μM for peptide P3 (SEQ ID NO: 2) and 25 μM for peptide P31 (SEQ ID NO: 1). The number of normal cells is presented as residual cells percentage (%) compared to the average of DMSO control. Whereas 100% represent no cytotoxicity or inhibition of cell growth, below 25/30% is considered cytotoxic and 0% represents acute cytotoxicity. This platform is equipped with Olympus right microscope (Spot NB camera and Simple PCI software, Compix), Right Zeiss Axiolmager M1 microscope (Marzhauser, Zeiss NB camera and AxioVision software) and the robots Arrayscan VTI Cellomics/Thermofisher, Hamilton Starlet, Hamilton Nimbus and Spotter Scienion.

### Results

### Antibiofilm Activity of the Peptides

This example demonstrates the prevention of biofilm formation by S. epidermidis ATTCC 35984.

The inventors demonstrated that the peptides of the present invention interfere with biofilm formation. The peptides at 1, 10, 25 or 100 μM were exposed to strong biofilm forming S. epidermidis RP62A (ATCC 35984). The remaining biofilm were quantified after 24 hours using crystal violet method. Biofilm decrease was observed at all tested concentrations. In particular, the peptide consisting of SEQ ID NO: 2 presented strong antibiofilm activity from 1 μM and more than 90% of biofilm reduction was detected at 10 μM.

They further demonstrated, surprisingly, that biofilm inhibition is not due to bactericidal activity. Accordingly, S. epidermidis colony forming units (CFU) was not affected in the presence of the peptide of SEQ ID NO: 2. Furthermore, the peptide has been localized by confocal fluorescence microscopy (CFM) images. The CFM images analysis show that the peptide does enter neither into bacterial cell nor into the wall or membrane but remains associated with extracellular matrix components.

In order to ensure the peptide future safe applicability, the inventors verified the biological cytotoxicity in 7 different mammalian lines (HuH7, CaCo-2, MDA, HCT116, PC3, NCI-H727 and MCF7), applying automated system image-based cellular content analysis (HCS/HCA). Thereby, cells treated with the peptide had exactly the same performance as untreated controls, evidencing absence of cytotoxicity.

As such, these results demonstrate that the peptides according to the invention may be used very effectively for preventing biofilm formation on a surface and acknowledge its safety by evidencing absence of cytotoxicity.

### Impairment of Initial Attachment, Aggregation and Biofilm Accumulation.

To reveal the peptide activity along the first stages of biofilm development, polystyrene coupons have been analyzed after 1, 4 and 24 hours of biofilm culture in presence or absence of the peptide. Scanning electron microscopy (SEM) analysis show bacteria attachment decreases after 1-hour of peptide exposition. Additionally, biofilm accumulation and cell aggregation profiles are strongly reduced after 4 and 24-hours demonstrating that the peptide prevents S. epidermidis coupons adherence. Notably this action remains after 24 h of incubation. The inventors further demonstrated, surprisingly, that antibiofilm mechanism of action is linked to extra cellular interactions independently of cell pressure regulation. The inventors selected some genes involved in different stages of biofilm development (atlE, aap, agrC, icaA, leuA, saeR, saeS, sarA, gyrB, rrsA) and analyzed its fold change by quantitative real-time PCR (qRT-PCR). Since exposed bacteria remain planktonic, the relative gene expression of them was compared to planktonic control cells. Exposed cells showed the same fold change as the planktonic control cells for all tested genes.

### Example 2

The present example demonstrates the antibiofilm activity of the peptides of the invention, in particular their capacity of preventing assembly of biofilm matrix.

### Materials and Methods

Peptides. All peptides were synthesized by Biomatik and ProteoGenix at purity grades over 95% in salts suitable for cell culture. For the assays, the peptides were all solubilized in ultra-pure sterile water.

Bacterial strains and growth conditions. Staphylococcus epidermidis ATCC 35984 was grown overnight on blood agar (Thermo Scientific Oxoid PB5039A) at 37° C. Oxoid LB agar was used for the colony-forming unit (CFU) assay. The other assays were done using a bacterial suspension of 3×10⁸ CFU/mL in tryptone soya broth (TSB, Oxoid) or 0.9% NaCl.

Biofilm formation. At least three technical and biological replicates were done for each assay of 1, 10, or 100 μM peptide concentrations.

Biofilm formation inhibition: A protocol adapted from Trentin et al. (2015) Scientific Reports 5 was used, with crystal violet in 96-well BD Falcon polyvinyl chloride (PVC) microtiter plates. The cell-bound stains were solubilized with absolute ethanol (Sigma-Aldrich), and absorbance was measured at 570 nm using a BIO-TEK PowerWave XS plate reader. The biofilm formation control represents 100% of biofilm formation.

Biofilm eradication: Biofilm was pre-formed as described above for 24 h at 37° C. without treatment. Afterwards, the wells were washed to remove planktonic cells, peptide solutions and controls were added, and all were incubated for 24 h. Biofilm eradication was verified by evaluating the remaining content by crystal violet.

### Bacterial Growth Assays.

Microtiter plates: Bacterial growth was evaluated by comparing OD600 values at the start and end of incubation in 96-well PVC microtiter plates.

Colony-forming units: After incubation at 37° C. for 24 h, CFU/mL was calculated to determine the peptide solution's bactericidal effects. The untreated growth control was considered to be 100% planktonic cells. At least three technical and biological replicates were performed for all assays.

Microscopic analysis. S. epidermidis ATCC 35984 biofilm was cultured as described above.

Scanning electron microscopy (SEM): Sterile 10×4 mm polystyrene coupons were inserted into bacterial cultures in the presence or absence of capsicumicine for 1, 4, and 24 h. The coupons were then washed with sterile 0.9% NaCl and fixed with 2.5% glutaraldehyde, 2% paraformaldehyde, and 0.1 M cacodylate buffer (pH 7.2). Afterwards, they were washed with 0.1 M cacodylate buffer and 0.2 M sucrose, then dehydrated with increasing concentrations of ethanol. A Leica EM CPD300 was used for critical point drying of the dehydrated samples. These were then sputtered with palladium in a Leica EM ACE200, and analyzed with a JEOL JSM-7100F microscope with EDS and EBSD at 10 kV.

Transmission electron microscopy (TEM): All well content was carefully detached at 1, 4, and 24 h, centrifuged at 10,000 g for 15 min at 4° C., then washed with sterile 0.9% NaCl. Fixation was performed at 4° C. with sodium 0.1 M cacodylate, 2% paraformaldehyde, 2.5% glutaraldehyde, and 75 mM lysine. Samples were washed with 0.1 M sodium cacodylate and 0.2 M sucrose, and contrasted with 1% osmium tetroxide and 1.5% potassium ferrocyanide. Dehydration was done with a gradual solution of ethanol and infiltration of increasing concentrations of LR White resin (Delta Microscopies, France). LR White resin inclusion and polymerization were then performed over 24 h at 60° C. in the absence of O₂. Thin 80 nm sections were collected onto carbon grids, and visualized at 200 kV with an FEI Tecnai Sphera microscope equipped with a Gatan 4 k×4 k CCD UltraScan camera.

Confocal fluorescence microscopy (CFM): Capsicumicine-fluorescein isothiocyanate (capsicumicine-ITC, 10 μM) was used to detect the capsicumicine peptide, whose antibiofilm activity was previously verified. After incubation for 1, 4, or 24 h, the well contents were carefully detached, centrifuged at 11,000 g for 2 min at 4° C., then washed with sterile 0.9% NaCl. The suspension was visualized directly or after adding 2 mg/mL Calcofluor White dye (Fluorescent Brightener 28, Sigma-Aldrich). To find bacterial cells permeated by the peptide control, the inventors used an FITC-labelled antimicrobial peptide (Pseudonajide, KRFKKFFMKLK-FITC (SEQ ID NO: 30)). Images were acquired via resonant scanner with a Leica SP8 DMI 6000 CS confocal microscope with hybrid detector, and ImageJ software was used for image analysis.

Quantitative reverse transcription PCR (qRT-PCR). After culturing for 24 h, RNAs were isolated from planktonic controls, biofilm controls, and from total cells exposed to 10 μM capsicumicine. An Invitrogen TRIzol Max bacterial RNA isolation kit and an Ambion TURBO DNase treatment were used as per manufacturer instructions. Total RNA concentrations and purities were assessed using a Biochrom SimpliNano spectrophotometer, and PCR reactions was done to ensure the complete absence of DNA. Each qRT-PCR reaction was then subjected to previously established quantities of cDNA (10 ng) and primers (0.2 μM). Reactional volumes were calculated using SYBR Select Master Mix (Applied Biosystems), as per the manufacturer's instructions. Primers were designed using the Primer3 program, then produced by Eurofins Genomics. Applied Biosystems StepOnePlus equipment and software were used. Relative transcript levels were determined by the ^{2-ΔΔct} method (Livak et al. (2001) Methods 25:402-408).

Real-time molecular self-assembly (RTMSA) assay. After checking the starting point (pH 7.2) of the assembly reaction for the synthetic staphylococcal matrix (Stewart et al. (2015) Sci. Rep. 5:13081), the inventors recorded the OD₆₀₀ as a function of the time every 30 sec until 30 min. Molecular self-assembly reactions were calculated to a final volume of 4 mL, with 0.3% chitosan (medium molecular weight, 75-85% deacetylation), 0.15% bovine serum albumin, and 0.015% lambda DNA (all from Sigma) in TSB. The concentration (μM) of tested peptides was calculated for a final volume of 4 mL. Before getting the assembly reaction pH starting points, a calibration record was done using the same reactional tube containing all reagents (auto zero). Acetic acid and NaOH were used to adjust pH, and the reaction temperature was about 30° C.

Cytotoxicity assay. Cytotoxicity assays were performed on the ImPACcell robotic platform (BIOSIT, Université de Rennes 1). Multiparameter high-content screening (HCS) and high-content analysis (HCA) were done on 7 different mammalian lines: HuH7, CaCo-2, MDA, HCT116, PC3, NCI-H727, and MCF7. The number of normal cells is presented as residual cell percentage compared to the DMSO control average.

### Results

Capsicumicine Prevents Biofilm Formation without Antibiotic Activity.

To begin, the inventors commercially synthesized three peptides of a natural fraction derived from Capsicum baccatum var. pendulum pepper seeds. P1 (RVQSEEGEDQISQRE, SEQ ID NO: 3), P2 (RAEAFQTAQALPGLCRI, SEQ ID NO: 4), and P3 (RSCQQQIQQAQQLSSCQQYLKQ, SEQ ID NO: 1) were the most stable fragments after chromatographic purification, enzymatic digestion and MALDI-MS fragmentation.

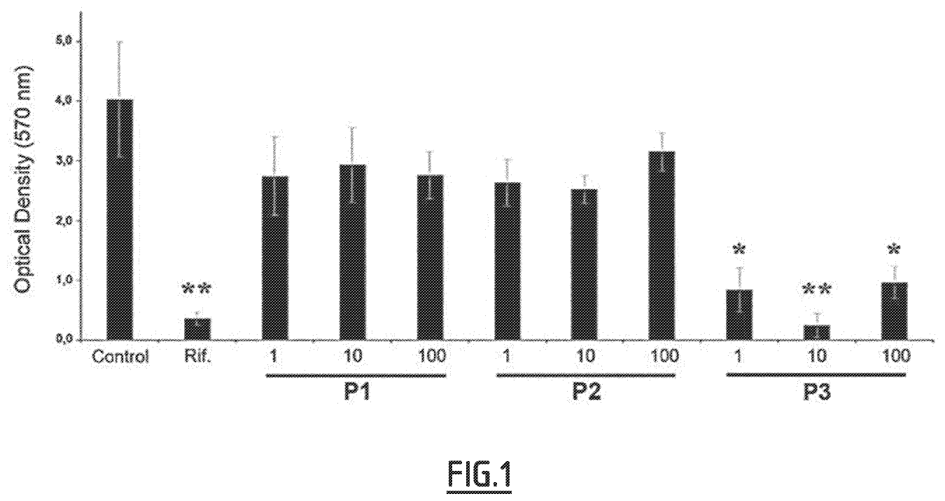

To find the most active one, the inventors exposed these compounds to strong biofilm-forming S. epidermidis RP62A (ATCC 35984). After 24 h, crystal violet was used to quantify the remaining biofilm, and P3, also called herein “capsicumicine,” was the most active, with particularly strong antibiofilm activity. Biofilm decreases were observed at all tested concentrations, but especially at 10 μM. There, biofilm was reduced by over 91%, independently of cell growth inhibition and not dependent on dose-response (FIG. 1).

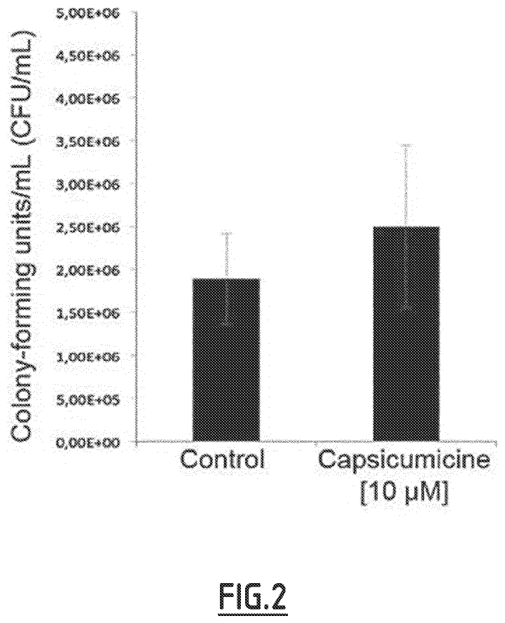

To examine the effects of capsicumicine on growth, the inventors checked S. epidermidis colony-forming unit (CFU) counts after peptide exposure. As expected, the CFUs were unchanged by capsicumicine, so the peptide's biofilm inhibition is not due to bactericidal activity (FIG. 2).

In order to localize the peptide, the inventors used capsicumicine conjugated to fluorescein isothiocyanate (capsicumicine-FITC) and confocal fluorescence microscopy (CFM). Analysis of the CFM images showed that capsicumicine-FITC stays associated with extracellular matrix components, not entering into bacterial cells or the walls or membranes (data not shown).

### Effects of Capsicumicine on the Eradication of Structured Biofilms.

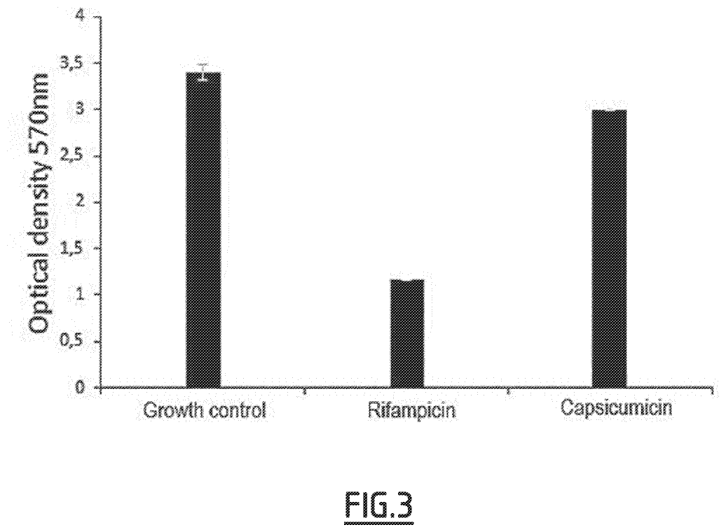

To verify the interactions between capsicumicine and established matrices, the inventors exposed a pre-existing S. epidermidis biofilm to a single concentration (100 μM) of the peptide, then after 24 h used crystal violet to quantify the total biomass. At that concentration, capsicumicine accounts for only about 15% of the disruption of pre-existing biofilm (FIG. 3).

### Capsicumicine is not Cytotoxic in Mammalian Cells.

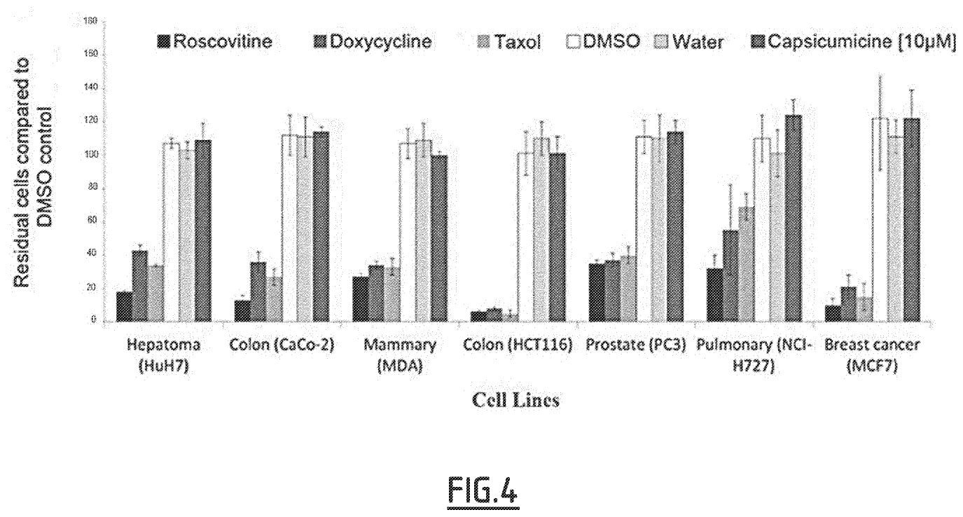

To ensure that capsicumicine is safe, the inventors verified its biological cytotoxicity in seven different representative human cell lines. They used automated image-based cellular content analysis, and found that capsicumicine-treated cells perform exactly the same as untreated controls, thus there is no cytotoxicity (FIG. 4).

### Independently of Cell Interactions, Capsicumicine Impairs Initial Biofilm Attachment, Aggregation, and Accumulation.

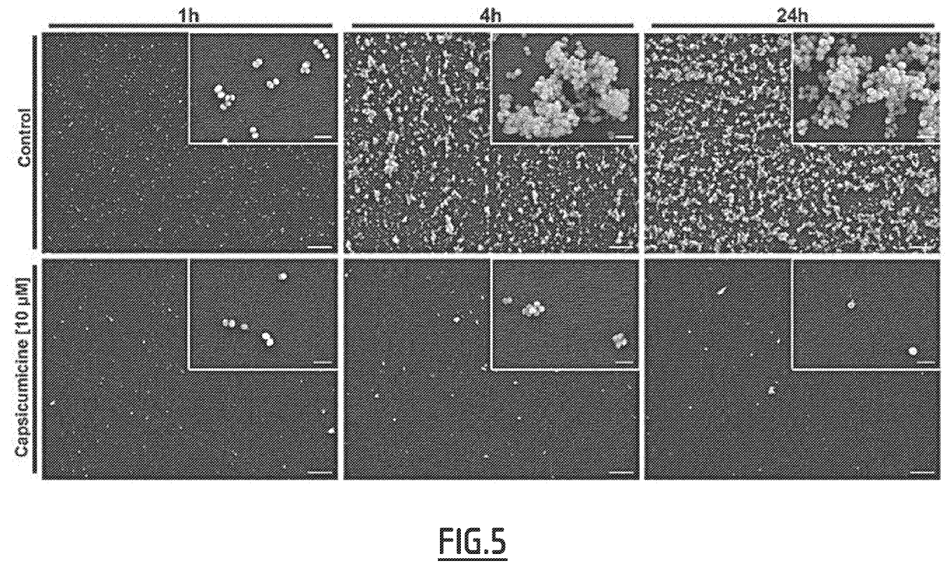

To explore its activity during the first stages of biofilm development, the inventors analyzed biofilm cultures on polystyrene coupons with and without capsicumicine after 1, 4, and 24 h. Scanning electron microscopy (SEM) analysis shows that bacterial attachment decreases after 1 h of capsicumicine exposure, with biofilm accumulation and cell aggregation profiles strongly reduced after 4 and 24 h (FIG. 5). This demonstrates that capsicumicine prevents S. epidermidis coupon adhesion, and notably this activity still occurs after 24 h incubation.

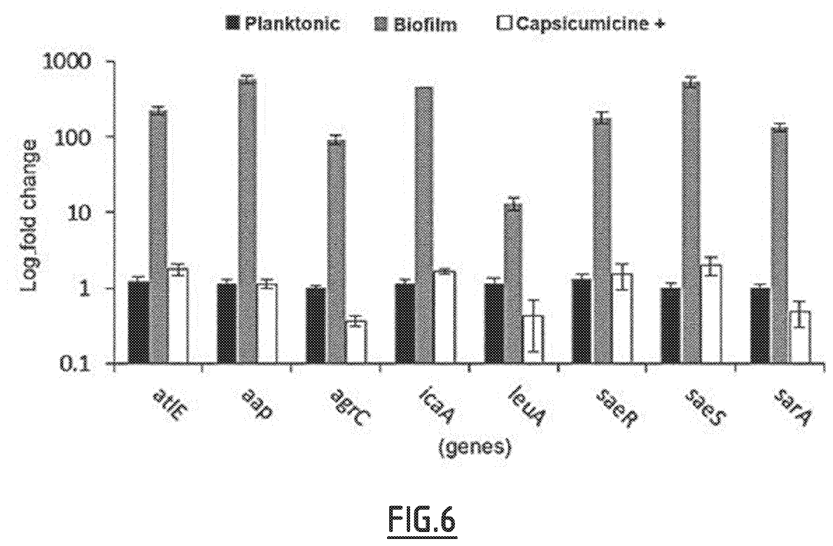

To study the peptide's possible mechanisms of action, the inventors selected several genes involved in different stages of biofilm development (atlE, aap, agrC, icaA, leuA, saeR, saeS, and sarA), with corresponding primers. Fold changes were analyzed by quantitative real-time PCR (qRT-PCR). Since exposed bacteria remain planktonic, the inventors compared their relative gene expressions to planktonic control cells. For all tested genes, capsicumicine-exposed cells show the same fold changes as the control (FIG. 6).

Capsicumicine Disturbs S. epidermidis Matrix Assembly.

Since capsicumicine's antibiofilm activity was not associated with direct bacterial interactions or gene expression modulation, the inventors used various microscopic approaches to investigate the interactions between the peptide and the extracellular matrix.

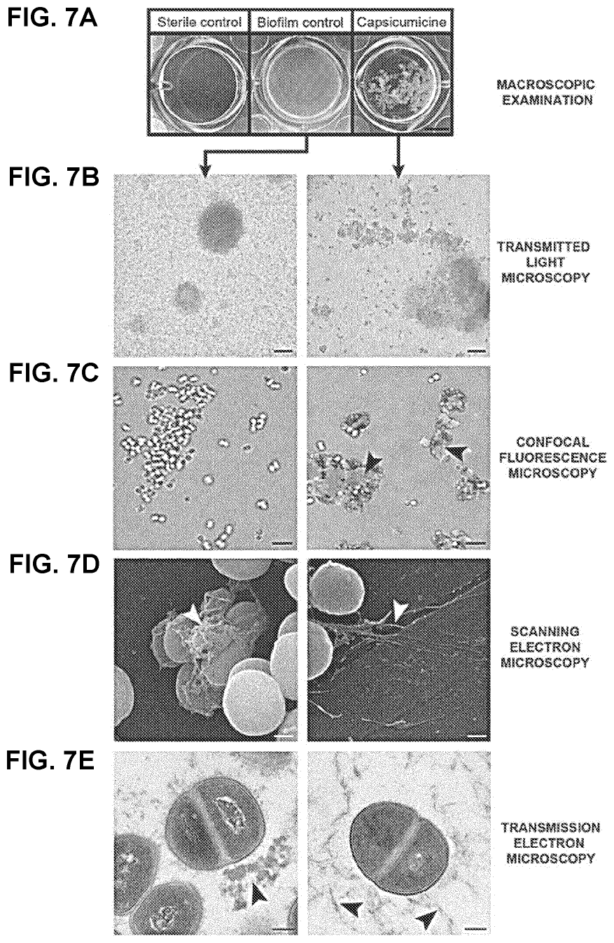

In the biofilm control, macroscopic observation showed a homogenous whitish adhered layer covering the walls and bottoms of the well (FIG. 7A, middle), but when capsicumicine was present, the inventors saw whitish flocculent non-adhered heterogeneous agglutinates (FIG. 7A, right).

Transmitted light microscopy images (FIG. 7B) matched these macroscopic observations. Similarly, CFM images confirmed the presence of labelled-capsicumicine in the matrix, which displayed a smooth cloud-like appearance (FIG. 7C). Crucially, scanning and transmission electron microscopy (SEM and TEM) techniques yielded ultra-structural descriptions that support these results, with the control biofilm matrix showing denser assembled globular-like structures, while the capsicumicine-exposed matrix was clearly less dense and had thin fibrillary branch-like structures (FIGS. 7D and E).

Therefore, different imaging techniques proved that matrix assembly changes when capsicumicine is present. Furthermore, since cellular morphologies were not different from the controls, the peptide and bacteria did not seem to interact.

### Capsicumicine Shifts the Molecular Self-Assembly of Artificial Matrices.

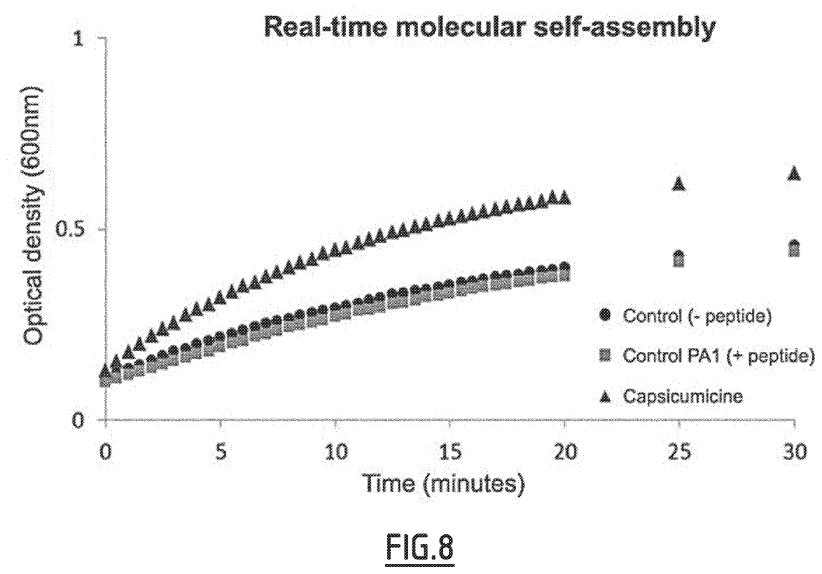

To confirm capsicumicine interactions with staphylococcal matrix assembly in the absence of metabolic or regulatory influences, the inventors used an artificial matrix model based on Stewart et al. (2015) Sci. Rep. 5:13051. Briefly, the inventors monitored the real-time molecular self-assembly reaction by measuring the optical density at 600 nm (OD₆₀₀) as a function of time with or without capsicumicine. As a negative control, the inventors used PA-1, a similarly sized peptide (Liu et al. (2016) Front Microbiol. 7:1228; Liu et al. (2017) Front Microbiol. 8:1766). OD increased when capsicumicine was present, which shows that the molecular self-assembly process is quicker overall (FIG. 8). Remarkably, the assembled matrix profiles were also visually different, with larger agglutinates in the presence of capsicumicine, although the matrices profiles for both controls were similar.

Capsicumicine Interacts with Exopolysaccharides.

To explore the peptide's potential affinities with these essential matrix components, the inventors exposed S. epidermidis cultures to capsicumicine, capsicumicine-FITC, and peptide antibiotic-FITC with calcofluor, then analyzed them all with CFM. They used calcofluor to target matrix polysaccharides and FITC for the peptides. The peptide control was an antibacterial peptide-FITC (Pseudonajide), and it showed green fluorescence in the cells but not in the matrix. CFM images showed substantial amounts of polysaccharides throughout the matrix when capsicumicine was present. Additionally, considerable amounts of capsicumicine-FITC appeared exclusively on the matrix.

### Conclusion

As shown here, capsicumicine, a peptide derived from Capsicum baccatum red pepper seeds, possesses strong antibiofilm activity. Capsicumicine prevents the establishment and maintenance of biofilm architecture through a new mechanism of action that is named here “matrix anti-assembly” (MAA).

MAA differs from matrix disassembly (Roy et al. (2018) Virulence 9:522-554) as instead of de-structuring the established matrix, it acts on the initial phase of assembly to prevent correct matrix assembly.

Bacterial surface proteins can passively interact with surfaces such as medical devices, generating an initial and reversible adhesion after electrostatic and hydrophobic interactions, Van der Waals forces, hydrodynamic forces, and so on (Speziale et al. (2014) Frot Cell Infect Microbiol. 4:171; Armbruster et al. (2018) Proc. Natl. Acad. Sci. USA 115:4317-4319; Even et al. (2017) Adv. Colloid. Interface Sci. 247:573-588). Bacteria will then require matrix production in order to remain attached after these weak interactions (Otto (2013) Annual Rev. Med. 64:175-188). During this process, physicochemical interactions drive molecular and colloidal matrix self-assembly, establishing a chain of dense architecture that results in stable adhesion (Dorken et al. (2012) J. R. Soc. Interface 9:3490-3502; Schwarz-Linek et al. (2012) Proc. Natl. Acad. Sci. USA. 109:4052-4057). In contrast, the peptide of the invention interacts with the extracellular matrix and modifies the self-assembly chain, resulting in a less dense and nonfunctional matrix, and impaired biofilm formation.

### Example 3

The present example demonstrated the antibiofilm activity of a number of peptides of the invention.

### Materials and Methods

Peptides. All peptides were synthesized by Biomatik and ProteoGenix at purity grades over 95% in salts suitable for cell culture. For the assays, the peptides were all solubilized in ultra-pure sterile water.

The tested peptides were as follows:

(SEQ ID NO: 1)
peptide P31 of sequence RSCQQQIQQAQQLSSCQQYLKQ-
RVQSEEGEDQISQRE,
(SEQ ID NO: 2)
peptide P3 of sequence RSCQQQIQQAQQLSSCQQYLKQ,
(SEQ ID NO: 3)
peptide P1 of sequence RVQSEEGEDQISQRE,
(SEQ ID NO: 6)
peptide P3a of sequence RSCQQQIQQAQQ,
(SEQ ID NO: 7)
peptide P3x of sequence RSCQQQ,
(SEQ ID NO: 8)
peptide P3y of sequence IQQAQQLS,
and
(SEQ ID NO: 9)
peptide P3z of sequence SCQQYLKQ.

Rifampicin was used as positive control.

### Bacterial Strains and Growth Conditions.

Staphylococcus epidermidis ATCC 35984 was grown overnight on blood agar (Thermo Scientific Oxoid PB5039A) at 37° C. Oxoid LB agar was used for the colony-forming unit (CFU) assay. The other assays were done using a bacterial suspension of 3×10⁸ CFU/mL in tryptone soya broth (TSB, Oxoid) or 0.9% NaCl.

Biofilm formation. At least three technical and biological replicates were done for each assay of 1, 10, 100, 200, 400 or 900 OA peptide concentrations.

Biofilm formation inhibition: A protocol adapted from Trentin et al. (2015) Scientific Reports 5 was used, with crystal violet in 96-well BD Falcon polyvinyl chloride (PVC) microtiter plates. The cell-bound stains were solubilized with absolute ethanol (Sigma-Aldrich), and absorbance was measured at 570 nm using a BIO-TEK PowerWave XS plate reader. The biofilm formation control represents 100% of biofilm formation.

### Bacterial Growth Assays.

Microtiter plates: Bacterial growth was evaluated by comparing OD600 values at the start and end of incubation in 96-well PVC microtiter plates.

Colony-forming units: After incubation at 37° C. for 24 h, CFU/mL was calculated to determine the peptide solution's bactericidal effects. The untreated growth control was considered to be 100% planktonic cells. At least three technical and biological replicates were performed for all assays.

### Results

Table 1 below shows the anti-biofilm activity of several peptides of the invention. The data presented here were selected based on the best activity observed at the lowest concentration.

TABLE 1
Antibiofilm activity evaluation of synthetic peptides
from Capsicum baccatum.

| | | Number | Biological activity | | |
|---|---|---|---|---|---|
| SEQ | | of amino | Concentration | Remanescent | Growth |
| ID | Peptide | acids | (μM) | biofilm (%) | (%) |
| 1 | P31 | 37 | 400 | 8.3 | 88.6 |
| 2 | P3 | 22 | 10 | 13.9 | 108.8 |
| 3 | P1 | 15 | 100 | 64.9 | 123.2 |
| 6 | P3a | 12 | 100 | 61.5 | 89.9 |
| 7 | P3x | 6 | 100 | 34.4 | 91.9 |
| 8 | P3y | 8 | 10 | 6.2 | 172.0 |
| 9 | P3z | 8 | 100 | 46.3 | 169.9 |

| | | | | |
|---|---|---|---|---|
| — | Rifampicin | 780 | 9.7 | 28.7 |

This example confirms the strong anti-biofilm activity of the peptides of the invention at concentrations as low as 10 μM.

1. A method for preventing biofilm formation by microorganisms on a surface, comprising the use of a peptide, wherein said peptide is
(i) a peptide consisting of 6 to 37, in particular 10 to 37, consecutive amino acids from a peptide of sequence SEQ ID NO: 1,
(ii) a peptide of sequence SEQ ID NO: 4, or
(iii) a peptidomimetic of (i) or (ii).

2. The method according to claim 1, wherein said peptide is a peptide consisting of an amino acid sequence selected from the group consisting of SEQ ID NOS: 2 and 3.

3. The method according to claim 1, wherein said peptide does not inhibit planktonic growth of the biofilm-forming microorganisms.

4. The method according to claim 3, wherein said microorganisms are antibiotic tolerant or antibiotic resistant bacteria.

5. The method according to claim 3, wherein said microorganisms are selected from the group consisting of Staphylococcus epidermis, Pseudomonas aeruginosa and Cryptococcus neoformans.

6. The method according to claim 1, wherein said surface is an abiotic or biotic surface.

7. An isolated peptide consisting of an amino acid sequence selected from the group consisting of sequences SEQ ID NO: 2 (RSCQQQIQQAQQLSSCQQYLKQ), SEQ ID NO: 1 (RSCQQQIQQAQQLSSCQQYLKQRVQSEEGEDQISQRE), SEQ ID NO: 3 (RVQSEEGEDQISQRE), SEQ ID NO: 4 (RAEAFQTAQALPGLCRI), SEQ ID NO: 5 (LSSCQQYLKQ), SEQ ID NO: 6 (RSCQQQIQQAQQ), SEQ ID NO: 7 (RSCQQQ), SEQ ID NO: 8 (IQQAQQLS) and SEQ ID NO: 9 (SCQQYLKQ), or a peptidomimetic thereof.

8. The isolated peptide according to claim 7, consisting of an amino acid sequence selected from the group consisting sequences SEQ ID NO: 2 (RSCQQQIQQAQQLSSCQQYLKQ), SEQ ID NO: 1 (RSCQQQIQQAQQLSSCQQYLKQRVQSEEGEDQISQRE), SEQ ID NO: 3 (RVQSEEGEDQISQRE) and SEQ ID NO: 4 (RAEAFQTAQALPGLCRI), or a peptidomimetic thereof.

9. A composition comprising the isolated peptide of claim 7.

10. The composition according to claim 9 wherein said peptide consists of an amino acid sequence which is SEQ ID NO: 2.

11. The composition according to claim 9 or 10, wherein the concentration of said peptide in the composition is 1-10 μM.

12. An ex vivo method for preventing biofilm formation by microorganisms on a device comprising at least one surface, said method comprising the step of coating said at least one surface with a composition according to claim 9.

13. The method according to claim 12, wherein said device is a medical or industrial device.

14. A device comprising at least one surface coated with the peptide as defined in claim 1.

15. A method for preventing infections in a patient by the prevention of biofilm formation by microorganisms, comprising administering to said patient a peptide as defined in claim 7.

16. The method according to claim 15, wherein said infection is endocarditis, osteomyelitis, chronic sinusitis, urethritis or periodontitis.
